(19) United States
(12) Patent Application Publication
(10) Pub. No.: US 2007/0275447 A1
(43) Pub. Date: Nov. 29, 2007

(54) Indirect or direct fermentation of biomass to fuel alcohol

(76) Inventors: Randy S. Lewis, Provo, UT (US); Ralph S. Tanner, Norman, OK (US); Raymond L. Huhnke, Stillwater, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(21) Appl. No.: 11/441,392

(22) Filed: May 25, 2006

(51) Int. Cl. C12P 7/06; C12N 1/20

(52) U.S. Cl. 435/161; 435/252.3

(57) ABSTRACT

A novel clostridia bacterial species (Clostridium carboxidivorans, ATCC BAA-624, “P7”) is provided. P7 is capable of synthesizing, from waste gases, products which are useful as biofuel. In particular, P7 can convert CO to ethanol. Thus, this novel bacterium can transform waste gases (e.g. syngas and refinery wastes) into useful products. P7 also catalyzes the production of acetate and butanol. Further, P7 is also capable of directly fermenting lignocellulosic materials to produce ethanol and other substances.

This invention was made using funds from grants from the United States Department of Agriculture Cooperative State Research, Education and Extension Service having grant numbers 2001-34447-10302, 2002-34447-11908, 2003-34447-13162, 2004-34447-14487, and 2005-34447-15711. The United States government may have certain rights in this invention.

### BACKGROUND OF THE INVENTION

1. Field of the Invention

The invention generally relates to bacteria that are capable of producing biofuel from waste. In particular, the invention provides a novel clostridia bacterial species (Clostridium carboxidivorans having the identifying characteristics of ATCC No. BAA-624) and a method of synthesizing ethanol and other useful products from CO using the clostridia species.

2. Background of the Invention

The development of renewable biofuels is a national priority motivated by both economic and environmental concerns, including reduction of greenhouse gas emissions, enhancement of the domestic fuel supply and maintenance of the rural economy. One promising avenue of development is the use of microbes to produce biofuel materials, particularly when the microbes do so by utilizing waste products generated by other processes, or low-cost agricultural raw material that can be locally produced.

Synthesis gas (“syngas”) is the major byproduct of the gasification of coal and of carbonaceous materials such as agricultural crops and residues. In contrast to combustion, which produces primarily CO₂ and water, gasification is carried out under a high fuel to oxygen ratio and produces largely H₂ and CO. Thus, syngas is composed largely of H₂ and CO, together with smaller amounts of CO₂ and other gases. Syngas can be used as a low-grade fuel; alternatively, it can be used in catalytic processes to generate a wide variety of useful chemical products, such as methane, methanol and formaldehyde (Klasson et al., 1992, Enz. Microb. Tech. 14: 602-608).

Anaerobic microorganisms such as acetogenic bacteria offer a viable route to convert syngas to useful products, in particular to liquid biofuels such as ethanol. Such bacteria catalyze the conversion of syngas with higher specificity, higher yields and lower energy costs than can be attained using chemical processes (Vega et al, 1990; Phillips et al., 1994). Several microorganisms capable of producing biofuels from waste gases and other substrates have been identified:

Three strains of acetogens (Drake, 1994) have been described for use in the production of liquid fuels from syngas: Butyribacterium methylotrophicum (Grethlein et al., 1990; Jain et al., 1994b); Clostridium autoethanogenum (Abrini et al., 1994); Clostridium ljungdahlii (Arora et al, 1995; Barik et al., 1988; Barik et al. 1990; and Tanner et al., 1993). Of these, Clostridium ljungdahlii and Clostridium autoethanogenum are known to convert CO to ethanol.

U.S. Pat. No. 5,173,429 to Gaddy et al. discloses Clostridium ljungdahlii ATCC No. 49587, an anaerobic microorganism that produces ethanol and acetate from CO and H₂O and/or CO₂ and H₂ in synthesis gas.

U.S. Pat. No. 5,192,673 to Jain et al. discloses a mutant strain of Clostridium acetobytylicum and a process for making butanol with the strain.

U.S. Pat. No. 5,593,886 to Gaddy et al. discloses Clostridium ljungdahlii ATCC No. 55380. This microorganism can anaerobically produce acetate and ethanol using waste gas (e.g. carbon black waste gas) as a substrate.

U.S. Pat. No. 5,807,722 to Gaddy et al. discloses a method and apparatus for converting waste gases into useful products such as organic acids and alcohols using anaerobic bacteria, such as Clostridium ljungdahlii ATCC No. 55380.

U.S. Pat. No. 6,136,577 to Gaddy et al. discloses a method and apparatus for converting waste gases into useful products such as organic acids and alcohols (particularly ethanol) using anaerobic bacteria, such as Clostridium ljungdahlii ATCC Nos. 55988 and 55989.

U.S. Pat. No. 6,136,577 to Gaddy et al. discloses a method and apparatus for converting waste gases into useful products such as organic acids and alcohols (particularly acetic acid) using anaerobic strains of Clostridium ljungdahlii.

U.S. Pat. No. 6,753,170 to Gaddy et al. discloses an anaerobic microbial fermentation process for the production of acetic acid.

Other strains of aceotgens have also been described for use in the production of liquid fuels from synthesis gas, e.g.: Butyribacterium methylotrophicum (Grethlein et al., 1990, Appl. Biochem. Biotech. 24/24:875-884); and Clostridium autoethanogenum (Abrini et al., 1994, Arch. Microbiol. 161:345-351).

For indirect fermentation methods, it is necessary to first convert a substrate to gases which are then utilized by microbes as described above. An alternative method is direct fermentation. In direct fermentation, the microbe catalyzes the production of products directly from the substrate; the step of converting the starting material to gas is not required, and both time and equipment costs can be substantially lowered. However, to date, no anaerobic bacteria have been identified that are capable of both indirect and direct fermentation of lignocellulosic material.

There remains an ongoing need to discover and develop additional microorganisms that are capable of producing useful products such as biofuels via fermentation. In particular, it would be advantageous to provide microbes that are robust, relatively easy to culture and maintain, and that provide good yields of products of interest, such as biofuels. Further, the prior art has failed to provide an anaerobic bacterium with the capacity to carry out both direct and indirect fermentation of lignocellulosic material.

### SUMMARY OF THE INVENTION

The present invention provides a novel biologically pure anaerobic bacterium, namely a strain of Clostridium carboxidivorans, ATCC BAA-624, deposited at the American Type Culture Collection in Manassas, Va., hereafter referred to as “P7” that is capable of producing high yields of valuable organic fluids from relatively common substrates. In particular, the microorganism can produce acetic acid, butyric acid, ethanol, butanol and other compounds by fermenting CO. One common source of CO is syngas, the gaseous byproduct of coal gasification. The microbes can thus convert substances that would otherwise be waste products into valuable products, some of which are biofuels. Syngas, and thus CO, can also be produced from readily available low-cost agricultural raw materials by pyrolysis, providing a means to address both economic and environmental concerns of energy production. The bacteria of the invention thus participate in the indirect conversion of biomass to biofuel via a gasification/fermentation pathway. Importantly however, P7 has also been found to have the ability to catalyze the direct fermentation of lignocellulosic material to produce, for example, ethanol and acetate.

Clostridium carboxidivorans can be used to produce butanol and butyric acid, in addition to ethanol and acetic acid. Cultures of Clostridium carboxidivorans are extremely stable and can be stored on the bench for over one year while retaining activity. Clostridium carboxidivorans is very tolerant of mishandling and upsets, especially exposure to oxygen (up to 2%). Clostridium carboxidivorans is the first anaerobe described capable of both direct and indirect fermentation of lignocellulosic biomass.

It is an object of this invention to provide a biologically pure culture of the microorganism Clostridium carboxidivorans. The microorganism has all of the identifying characteristics of ATCC No. BAA-624.

In addition, the invention provides a composition for producing ethanol. The composition comprises 1) a source of CO, and 2) Clostridium carboxidivorans. In one embodiment of the invention, the source of CO is syngas.

In yet another embodiment, the invention provides a method of producing ethanol. The method comprises the step of combining a source of CO and Clostridium carboxidivorans under conditions which allow said Clostridium carboxidivorans to convert CO to ethanol.

The invention further provides a system for producing ethanol, the system comprising 1) a vessel in which a source of CO is combined with Clostridium carboxidivorans; and 2) a controller which controls conditions in said vessel which permit the Clostridium carboxidivorans to convert the CO to ethanol. In one embodiment of the invention, the system also includes 1) a second vessel for producing syngas; and 2) a transport for transporting the syngas to the vessel, wherein the syngas serves as the source of CO. Such a system is illustrated in FIG. 7, which shows the vessel 100 and controller 101, with the optional second vessel 200 and transport 201.

The invention further provides a method for the direct fermentation of lignocellulosic biomass. The method comprises the step of combining a source of lignocellulosic biomass and Clostridium carboxidivorans under conditions which allow the Clostridium carboxidivorans to directly ferment the lignocellulosic biomass. Ethanol and/or acetic acid are among the products that are produced by this direct fermentation reaction.

### BRIEF DESCRIPTION OF THE DRAWINGS

FIGS. 1A and B. Culture of P7. A, cell concentration (absorbance at 600 nm) vs time (days); B, culture temperature (° F.) vs time (days).

FIGS. 2A-C. Culture of P7. A, CO profile vs time (days); B, CO₂ profile vs time (days); C, cell concentration (absorbance at 600 nm) vs time (days).

FIGS. 3A and B. Culture of P7. A, cell concentration (absorbance at 600 nm) vs time (days); B, pH of culture medium vs time (days).

FIGS. 4A-C. Culture of P7. A, CO profile vs time (days); B, CO₂ profile vs time (days); C, cell concentration (absorbance at 600 nm) vs time (days).

FIG. 5. Gas chromatogram showing production of ethanol and butanol by P7.

FIGS. 6A-E. Bubble column bioreactor experimental results obtained with novel clostridia bacterium, P7. A, cell concentration vs time; B, CO utilization vs time; C, ethanol, butanol and acetate formation with time; D, yield of cells per mole of CO; E, yield of ethanol per mole of CO.

FIG. 7. Schematic representation of a system for producing ethanol according to the invention.

### DETAILED DESCRIPTION OF THE PREFERRED EMBODIMENTS OF THE INVENTION

The present invention is based on the discovery of a novel acetogenic bacterium that is capable, under anaerobic conditions, of producing high yields of valuable products from CO and other readily available substrates. In particular, the microorganism produces valuable liquid products such as ethanol, butanol and acetate by fermenting CO, with ethanol being a predominant product. By “fermenting” we mean a physiological process whereby the substrate serves as both the source of electrons and the electron sink (oxidation of a portion of the substrate and reduction of a portion of the substrate) which can be used for the production of products such as alcohols and acids. As a result, this organism is capable of converting what would otherwise be waste gases into useful products such as biofuel. The anaerobic microbe of the invention is a novel clostridia species which displays the characteristics of purified cultures represented by ATCC deposit BAA-624, herein referred to as “P7”.

The morphological and biochemical properties of P7 have been analyzed and are described herein in the Examples section below. While certain of the properties of P7 are similar to other Clostridium species, P7 possesses unique characteristics that indicate it is a novel species of this genus. P7 has been denominated Clostridium carboxidivorans, and is considered to be representative of this species.

The bacteria in the biologically pure cultures of the present invention have the ability, under anaerobic conditions, to produce ethanol from the substrates CO+H₂O and/or H₂+CO₂ according to the following reactions:

### Ethanol Synthesis

6CO+3H₂O→C₂H₅OH+4CO₂ (1)

6H₂+2CO₂→C₂H₅OH+3H₂O (2)

With respect to the source of these substrates, those of skill in the art will recognize that many sources of CO, CO₂ and H₂ exist. For example, preferred sources of the substrates are “waste” gases such as syngas, oil refinery waste gases, gases (containing some H₂) which are produced by yeast fermentation and some clostridial fermentations, gasified cellulosic materials, coal gasification, etc. Alternatively, such gaseous substrates are not necessarily produced as byproducts of other processes, but may be produced specifically for use in the fermentation reactions of the invention, which utilize P7. Those of skill in the art will recognize that any source of substrate gas may be used in the practice of the present invention, so long as it is possible to provide the bacterium with sufficient quantities of the substrate gases under conditions suitable for the microbe to carry out the fermentation reactions. The source of H₂O for the reaction represented by Equation (1) is typically the aqueous media in which the organism is cultured.

In a preferred embodiment of the invention, the source of CO, CO₂ and H₂ is syngas. Syngas for use as a substrate may be obtained, for example, as a gaseous byproduct of coal gasification. The bacteria thus convert a substance that would otherwise be a waste product into valuable biofuel. Alternatively, syngas can be produced by gasification of readily available low-cost agricultural raw materials expressly for the purpose of bacterial fermentation, thereby providing a route for indirect fermentation of biomass to fuel alcohol. There are numerous examples of raw materials which can be converted to syngas, as most types of vegetation could be used for this purpose. Preferred raw materials include but are not limited to perennial grasses such as switchgrass, crop residues such as corn stover, processing wastes such as sawdust, etc. Those of skill in the art are familiar with the generation of syngas from such starting materials. In general, syngas is generated in a gasifier from dried biomass primarily by pyrolysis, partial oxidation, and steam reforming, the primary products being CO, H₂ and CO₂. (The terms “gasification” and “pyrolysis” refer to similar processes. Both processes limit the amount of oxygen to which the biomass is exposed. Gasification allows a small amount of oxygen (this may also be referred to as “partial oxidation” and pyrolysis allows more oxygen. The term “gasification” is sometimes used to include both gasification and pyrolysis.) Typically, a part of the product gas is recycled to optimize product yields and minimize residual tar formation. Cracking of unwanted tar and coke in the syngas to CO may be carried our using lime and/or dolomite. These processes are described in detail in, for example, Reed, 1981. (Reed, T. B. (1981) Biomass gasification: principles and technology, Noves Data Corporation, Park Ridge, N.J.)

In addition, combinations of sources of substrate gases may be utilized. For example, the primary source of CO, CO₂ and H₂ may be syngas, but this may be supplemented with gas from other sources, e.g. from various commercial sources. For example, the reaction according to Equation (1) above generates four molecules of CO₂, and reaction according to Equation (2) utilizes 6 H₂ but only two molecules of CO₂. Unless H₂ is plentiful, CO₂ buildup may occur. However, supplementing the media with additional H₂ would result in an increase of the utilization of CO₂, and the consequent production of yet more ethanol. While a primary product produced by the fermentation of CO by the bacterium of the present invention is ethanol, other useful liquid products are also produced. In the Examples section below, the production of acetate and butanol from CO+H₂O and H₂+CO₂ is also documented. Acetate production likely occurs via the following reactions:

### Acetate Synthesis

4CO+2H₂O→CH₃COOH+CO₂ (3)

4H₂+2CO₂→CH₃COOH+2H₂O (4)

while butanol production likely occurs via the following reactions:

### Butanol Synthesis

12 CO+5 H₂O→C₄H₉OH+8CO₂

12H₂+4CO₂→C₄H₉OH+7 H₂O.

The organisms of the present invention must be cultured under anaerobic conditions. By “anaerobic conditions” we mean that dissolved oxygen is absent from the medium.

In general, the media for culturing the acetogen of the invention is a liquid medium such as ATCC medium 1754 (developed by R. S. Tanner). However, those of skill in the art will recognize that alternative media can be utilized, for example, ATCC medium 1045 under a H₂:CO₂ or CO:CO₂ atmosphere at an initial pH of 6. Further, various media supplements may be added for any of several purposes, e.g. buffering agents, metals, vitamins, salts, etc. In particular, those of skill in the art are familiar with such techniques as nutrient manipulation and adaptation, which result in increased or optimized the yields of a product of interest. For example, culturing microbes under “non-growth” conditions (i.e. conditions which do not favor bacterial growth and reproduction) may result in higher production of fermentation products. This is likely because under non-growth conditions, the resources of the bacteria are not dedicated to reproduction and are therefore free for other synthetic activities. Examples of non-growth conditions include, for example, maintaining the culture at non-optimal temperature or pH, the limitation of nutrients and carbon sources, etc. Generally, non-growth conditions would be implemented after a desired density of bacteria is reached in the culture. Also, it is possible by media optimization to favor production of one product over others, e.g. to favor the production of ethanol over acetate and butanol. For example, increasing the concentration of iron tenfold compared to that in standard medium doubles the concentration of ethanol produced, while decreasing the production of acetic acid and butyric acid. Those of skill in the art are familiar with procedures for optimizing the production of desired products, and all such optimized procedures using the P7 bacterium are intended to be encompassed by the present invention. Reference is made, for example, to work carried out with Clostridium acetobutylicum which provides guidance for such techniques (see, for example, Bahl et al., 1986, Appl Environ. Microbiol. 52:169-172; and U.S. Pat. No. 5,192,673 to Jain et al. and U.S. Pat. No. 5,173,429 to Gaddy, the complete contents of both of which are hereby incorporated by reference).

In particular, Clostridium carboxidivorans may be cultured using Balch technique (Balch and Wolfe, 1976, Appl. Environ. Microbiol. 32:781-791; Balch et al., 1979, Microbiol. Rev. 43:260-296), as described in the reviews by: Tanner, 1997, Manual Environ. Microbiol., p. 52-60, ASM Press; Tanner, 2002, Manual Environ. Microbiol. 2nd ed., p. 62-70; Wiegel et al., 2005, An Introduction to the Family Clostridiaceae, The Prokaryotes, Release 3.20; Tanner, 2006, Manual Environ. Microbiol. 3rd ed., ASM Press. This entails the aid of an anaerobic chamber for preparing culture materials and a gas exchange manifold to establish whatever gas phase is desired for culture in sealed tubes or vessels. More specific details on culturing solvent-producing acetogens, such as the use of an acidic pH, appear in Tanner et al., 1993, Int. J. Syst. Bacteriol. 43:232-236 and Liou et al., 2005, Int. J. Syst. Evol. Microbiol. 55:2085-2091. Methods to enhance ethanol production include optimization of every medium component (such as ammonium, phosphate and trace metals), control of culture pH, mutagenesis and clonal screening etc.

The fermentation of CO by the organisms of the invention can be carried out in any of several types of apparatuses that are known to those of skill in the art, with or without additional modifications, or in other styles of fermentation equipment that are currently under development. Examples include but are not limited to bubble column reactors, two stage bioreactors, trickle bed reactors, membrane reactors, packed bed reactors containing immobilized cells, etc. The chief requirements of such an apparatus include that sterility, anaerobic conditions, and suitable conditions or temperature, pressure, and pH be maintained; and that sufficient quantities of substrates are supplied to the culture; that the products can be readily recovered; etc. The reactor may be, for example, a traditional stirred tank reactor, a column fermenter with immobilized or suspended cells, a continuous flow type reactor, a high pressure reactor, a suspended cell reactor with cell recycle, and other examples as listed above, etc. Further, reactors may be arranged in a series and/or parallel reactor system which contains any of the above-mentioned reactors. For example, multiple reactors can be useful for growing cells under one set of conditions and generating ethanol (or other products) with minimal growth under another set of conditions.

In general, fermentation will be allowed to proceed until a desired level of product is produced, e.g. until a desired quantity of ethanol is produced in the culture media. Typically, this level of ethanol is in the range of at least about 1 gram/liter of culture medium to about 50 gram/liter, with a level of at least about 30 gram/liter (or higher) being preferable. However, cells or cell culturing systems that are optimized to produce from about 1 to 10, or from about 10 to 20, or from about 20 to 30, or from about 30 to 40, or from about 40 to 50 gram/liter are also contemplated. P7 remains viable and will grow in ethanol concentrations of at least 60 g/L. Alternatively, production may be halted when a certain rate of production is achieved, e.g. when the rate of production of a desired product has declined due to, for example, build-up of bacterial waste products, reduction in substrate availability, feedback inhibition by products, reduction in the number of viable bacteria, or for any of several other reasons known to those of skill in the art. In addition, continuous culture techniques exist which allow the continual replenishment of fresh culture medium with concurrent removal of used medium, including any liquid products therein (i.e. the chemostat mode).

The products that are produced by the bacteria of the invention can be removed from the culture and purified by any of several methods that are known to those of skill in the art. For example, ethanol can be removed and further processed, e.g. by solvent extraction; distillation to the azeotrope followed by azeotropic distillation; molecular sieve dehydration; pervaporation; or flow-through zeolite tubes. Those of skill in the art will recognize that the two main techniques in industry for ethanol dehydration following distillation are azeotropic distillation and molecular sieve dehydration. (See, for example, Kohl, S. “Ethanol 101-7: Dehydration” in Ethanol Today, March 2004: 40-41). In addition, depending on the number of products, several separation techniques may need to be employed to obtain several pure products. Likewise, acetate and butanol may be removed and further processed by similar processes.

In some embodiments of the invention, P7 is cultured as a pure culture in order to produce ethanol (or other products of interest). However, in other embodiments, P7 may be cultured together with other organisms.

Another additional point of novelty for the present invention is the discovery that P7 is capable of directly fermenting lignocellulosic biomass. In other words, in order for P7 to produce useful products as described herein, is it not necessary to first gasify the substrate, (for example, to gasify a lignocellulosic material such as plant material (e.g. switchgrass) to produce CO). Rather, P7 is able to produce the useful products via direct fermentation of the lignocellulosic biomass. P7 is the first anerobe known to have this capability. The invention thus also includes a method for the direct fermentation of lignocellulosic material. The method involves the step of combining a source of lignocellulosic biomass and Clostridium carboxidivorans under conditions which allow the bacterium to directly ferment the lignocellulosic biomass. Ethanol and/or acetic acid are exemplary products of the direct fermentation of lignocellulosic biomass by Clostridium carboxidivorans.

### EXAMPLES

The development of renewable biofuels is a national priority motivated by both economic and environmental concerns, including reduction of greenhouse gas emissions, enhancement of domestic fuel supply and maintenance of the rural economy. Preliminary research on the fermentation of CO to ethanol has yielded the following. A novel acetogen was isolated from an agricultural lagoon based on its ability to produce ethanol from CO. The acetogen was selected for further strain development because of its very stable culture and storage characteristics. A four-liter, bubble column bioreactor was built and control of key fermentation parameters established, including sterility, anaerobiosis, temperature and pH.

### Introduction

The combustion of carbonaceous materials, such as agricultural crops and residues, under controlled conditions produces synthesis gas. Synthesis gas (syngas) is composed mainly of carbon monoxide, carbon dioxide and hydrogen. Syngas can be directly used in catalytic processes to generate a wide variety of chemicals, such as methane, methanol and formaldehyde or used as a low-grade fuel (Klasson ct al., 1992). Anaerobic bacteria, capable of autotrophic growth, offer an alternate route to convert syngas to liquid biofuels, such as ethanol, at higher specificity, higher yields and lower energy costs than chemical processes at ambient conditions of temperature and pressure (Vega et al., 1990, Phillips et al., 1994).

Development of liquid biofuels based on low-cost agricultural raw materials would benefit the US by reducing the nation's dependence on imported oil from politically unstable, mid-east countries (Barfield et al., 1997). Other advantages of biofuels include environmental concerns, such as the greenhouse effect and net atmospheric carbon balance, and development of rural economy. A holistic approach to biofuel generation may include the following steps:
1) Harvest and storage of agricultural crops, of which switchgrass is the model crop, from native grasslands.
2) Gasification of dried switchgrass in a fluidized-bed reactor to generate syngas and downstream processing of syngas to eliminate deleterious compounds such as tar, ash, etc.
3) Microbial conversion of purified syngas to ethanol under anaerobic conditions in a reactor, e.g. a bubble column bioreactor.

Evaluation of production, harvest, transportation, storage and processing of agricultural crops has been performed. This includes determination of the crop quality and composition by chemical analysis, estimation of transportation and storage costs, and breeding and screening of new crop varieties to improve biomass yield per acre (Taliaferro et al., 1975, Huhnke and Bowers, 1994).

Syngas can be generated, for example, in a gasifier from dried biomass primarily by pyrolysis and partial oxidation. A part of the product gas can be recycled to optimize product yields and minimize residual tar formation. Cracking of unwanted tar and coke in the syngas to CO can be accomplished using lime and/or dolomite in the gasifier. Gas purification strategies to provide a quality gas-feed to the bioreactor can be optimized.

### EXAMPLE 1

### Identification and Initial Characterization of P7

### Isolation of P7

According to the present invention, the microbial catalyst used to convert syngas to liquid products (such as ethanol, butanol and acetate) is a novel acetogen, P7, which was isolated from an agricultural settling lagoon located in Oklahoma. P7 was isolated by methods that are known by those of skill in the art. Briefly, inocula were used to set up enrichments in a mineral medium (Tanner, 1997, in Manual of Environmental Microbiology, Hurst et al., eds. ASM Press, Washington D.C.) supplemented with yeast extract and incubated at both 37° C. and 50° C. in the presence and absence of BESA (an inhibitor of methogens but not acetogens) and under a CO:CO₂:N₂ (60:30:10) atmosphere. Enrichments were monitored for gas consumption, ethanol production and acetic acid production. Ethanol producing enrichments were further incubated at 37° C. Enrichments showed a decrease in culture pH from an initial pH of 6.0 to a final pH of 4-5. Microscopic observation and final culture pH both indicated that purified P7 from one such enrichment differs from other known ethanol producing organisms (e.g. Butyribacterium methylotropicum, Clostridium autoethanogenum and Clostridium ljungdahlii. General methods for the isolation and initial culturing of bacteria are outlined, for example, in Bryant, 1972 (Am Journ Clin Nutrition 25, 1324-1328).

### Determination of Culture and Storage Characteristics

Once purified, P7 was maintained as a biologically pure culture in the laboratory under the following conditions: P7 was routinely maintained by transferring into fresh medium every 1-2 weeks. Cultures can, however, be stored on the bench for over a year. For longer term storage, cultures can be lyophilized and frozen, or stored in 50% glycerol at −20° C. Such techniques for the storage and handling of anaerobic bacteria are described, for example, in Sower and Schreier (1995, Archea, A Laboratory Manual, Methanogens, Cold Spring Harbor Press).

During the culture and storage of P7, it was observed that this organism displayed exceptionally stability, robustness, and flexibility. For example, as noted above, cultures are stable on the bench at room temperature for extended periods of time. Cultures of P7 can recover from an exposure to 2% oxygen in the gas phase and continue to produce ethanol from carbon monoxide during recovery. P7 cultures exhibited the ability to resume initial performance following major changes in selected critical operating parameters (e.g. pH, temperature, etc.). In addition, cultures of P7 reach a cell density of 1 O.D. units in a short period of time (e.g. about 24 hours) and the P7 culture does not readily lyse out. Further, P7 cultures are capable producing promisingly high levels of ethanol (see below).

### Characterization of P7

P7 was characterized as a separate, novel species of the clostridial rRNA homology group 1. For example, FAME (fatty acid methyl ester) analysis showed that strain P7 is different from C. ljungdahlii by at least 30 euclidean distance units (not shown). For comparison, the two distinct species Clostridium butyricum and Clostridium acetobutylicum showed a difference of only about 10 euclidean distance units between them. (The greater the distance, the more different the FAME profiles.) P7 was also shown to be a distinct species by 16S rRNA gene analysis and by DNA reassociation analysis (Liou et al, 2005, Int. J. Syst. Evol. Micorbiol. 55:2085-2091) (not shown).

### Experiments with Trace Metal Concentration

Initial cultures of P7 were established in a bioreactor with the following medium: 20 ml/l minerals, 10 ml/l vitamins, and 5 ml/l trace metals. The precise compositions of these ingredients are given in Tables 1, 2 and 3, respectively.

TABLE 1
Mineral solution^{a}

| Component | Amt (g)/liter |
|---|---|
| NaCl | 80 |
| NH₄Cl | 100 |
| KCl | 10 |
| KH₂PO₄ | 10 |
| MgSO₄•7H₂O | 20 |
| CaCl₂•2H₂O | 4 |

^{a}A solution containing the major inorganic components required for microbial growth. Add and dissolve each component in order. The mineral solution can be stored at room temperature.

TABLE 2
Vitamin solution^{a}

| Component | Amt (mg)/liter |
|---|---|
| Pyridoxine•HCl | 10 |
| Thiamine•HCl | 5 |
| Riboflavin | 5 |
| Calcium pantothenate | 5 |
| Thioctic acid | 5 |
| p-Aminobenzoic acid | 5 |
| Nicotinic acid | 5 |
| Vitamin B₁₂ | 5 |
| MESA^{b} | 5 |
| Biotin | 2 |
| Folic acid | 2 |

^{a}A solution designed to meet the water-soluble vitamin requirements of many microorganisms. Store at 4° C. in the dark.
^{b}Mercaptoethanesulfonic acid.

TABLE 3
Trace metal solution^{a}

| Component | Amt (g)/liter |
|---|---|
| Nitrilotriacetic acid | 2.0 |
| Adjust pH to 6 with KOH | |
| MnSO₄•H₂O | 1.0 |
| Fe(NH₄)₂(SO₄)₂•6H₂O | 0.8 |
| CoCl₂•6H₂O | 0.2 |
| ZnSO₄•7H₂O | 0.2 |
| CuCl₂•2H₂O | 0.02 |
| NiCl₂•6H₂O | 0.02 |
| Na₂MoO₄•2H₂O | 0.02 |
| Na₂SeO₄ | 0.02 |
| Na₂WO₄ | 0.02 |

^{a}A solution designed to meet the trace metal requirements of many microorganisms. Store at 4° C.

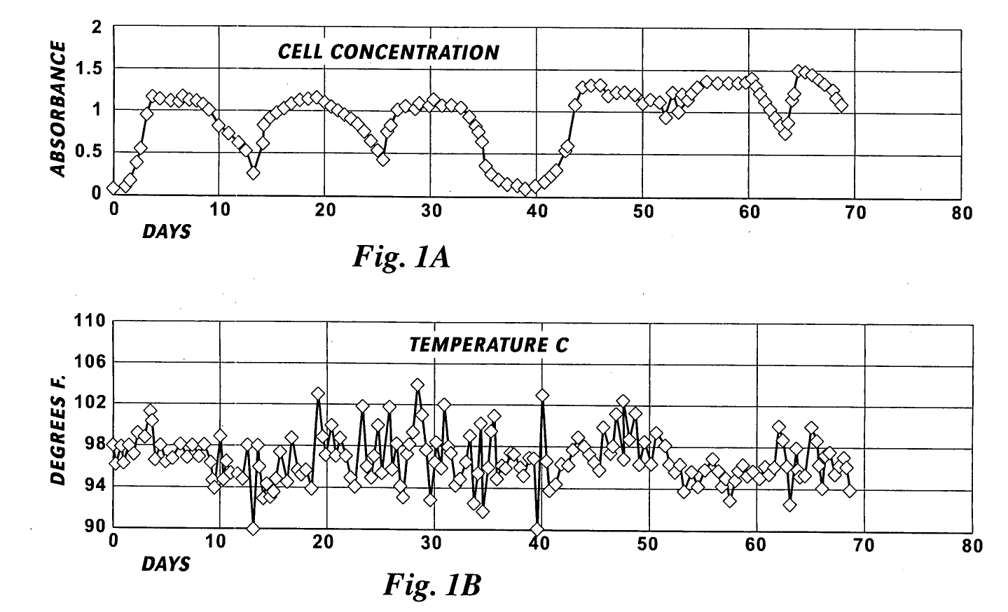

Gas feed to the bioreactor consisted of 60% nitrogen, 25% CO and 15% CO₂. 5 g/l of MES (2-(N-morpholino)ethanesulfonic acid) buffer and 0.5 g/l of yeast extract were added. As can be seen in FIG. 1A, the cells were relatively unstable in this medium, requiring the replacement of media on days 13, 25, 40, 52 and 63 of the 70 day experiment. FIG. 1B shows the temperature of the culture over the course of this experiment.

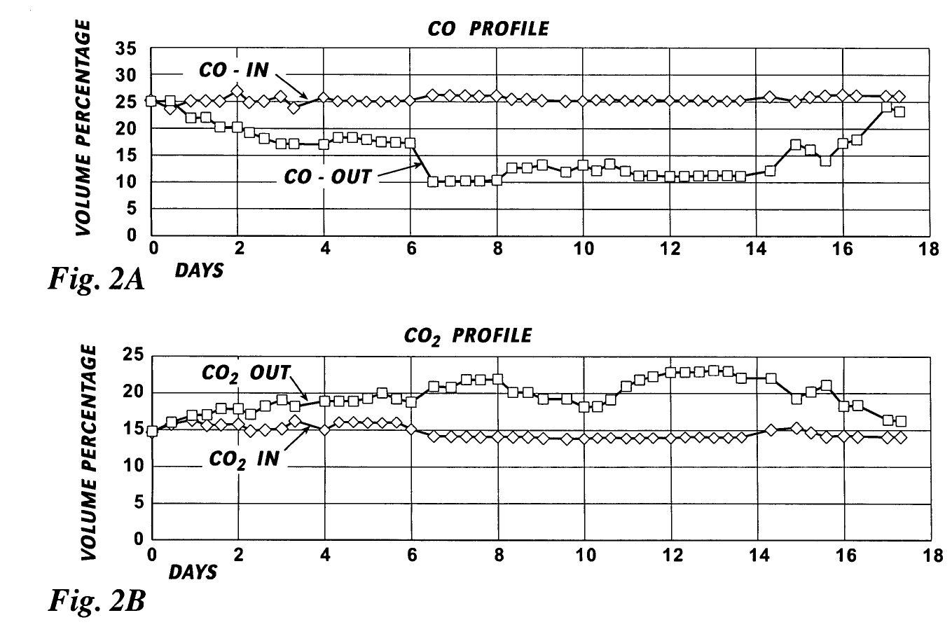

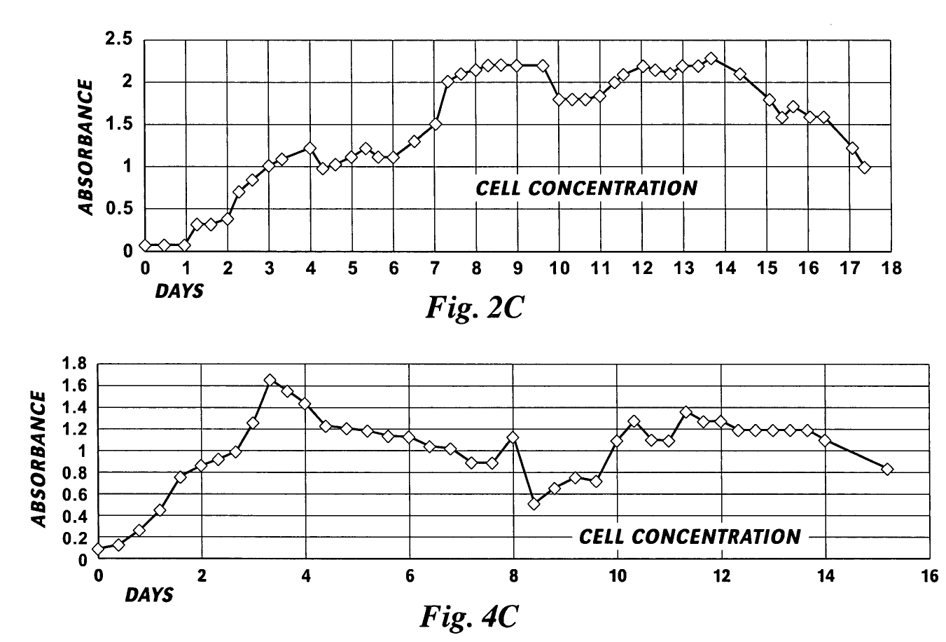

To improve the cell concentration and maintain cell stability, the trace metal concentration was doubled (i.e. to 10 ml/l) on day 6 of the experiment. As can be seen in FIG. 2C, this resulted in an increase in OD from about 1.1 to about 2.2 by day 8. FIGS. 2A and 2B show the culture's CO and CO₂ profiles, respectively, throughout the experiment. Subsequently, on day 13, the iron content of the trace metals was reduced to 50% of the initial concentration. This resulted in a steady drop in OD until termination of the experiment at day 17. This result demonstrates that media manipulation plays a key role in the cell OD and that the iron content is a significant component. Media manipulation is a common technique known to those of skill in the art.

Additional experimentation showed that adding sodium sulfide to the culture medium also improved cell stability. Initially the medium was inoculated with 4 ml of 5 wt % sodium sulfide per liter of medium. As the cell concentration increased, the sulfide concentration was observed to drop below 0.1 ppm, and the OD of the culture also decreased. Therefore, the sulfide concentration in the bioreactor was maintained between 0.1 and 1 ppm by adding sodium sulfide as needed. Under these circumstances, the OD increased to 1.7 and remained stabile, unlike the cycling observed in FIG. 1A in the absence of sodium sulfide.

### Requirement for CO₂

Experiments were conducted to assess the requirement for CO₂ for culturing P7. The media that was utilized was the same used for the trace metal concentration studies, and the liquid volume in the bioreactor was 4.5 liters. Cell concentration in the bioreactor was controlled by operating the bioreactor without a product filter in a chemostat-mode. Initially, the bioreactor was batch-operated with response to the liquid feed and switched to a continuous mode to maintain the cell concentration at lower values (at least 50%) compared to earlier runs. Dilution rate was varied at 2 ml/min and 4 ml/min. The gas flow rate was maintained at 200 ccm. To study the effect of CO₂, the gas compositions was set at 75% N₂ and 25% CO for the first runs, and 60% N₂, 25% CO and 15% CO₂ for later runs.

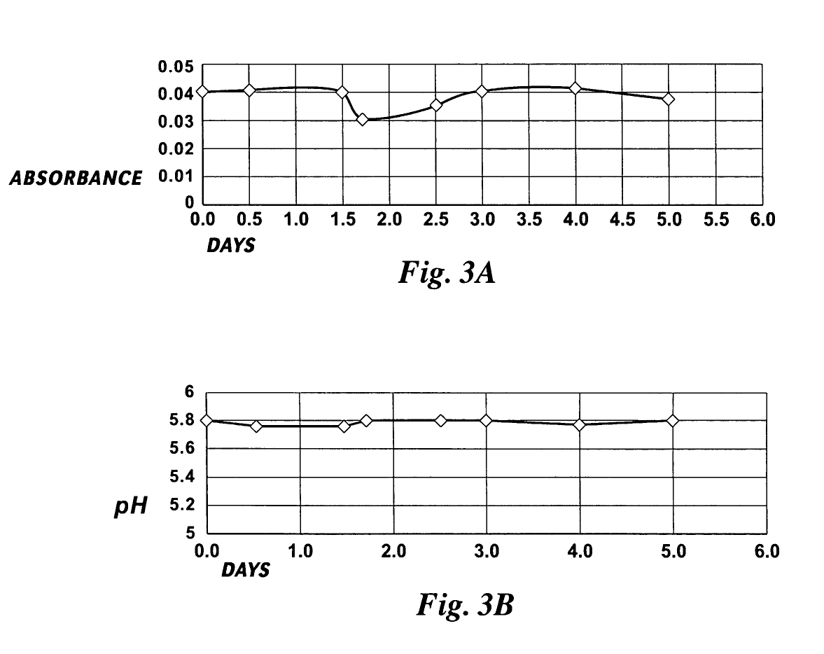

FIGS. 3A and B show the results of a 5 day attempt to culture P7 under the conditions described above, but in the absence of added CO₂. As can be seen in FIG. 3A, in the absence of CO₂ no appreciable cell growth was observed even with a week-long exposure. This established the necessity of CO₂ for cell growth FIG. 3B shows the pH of the culture during the experiment.

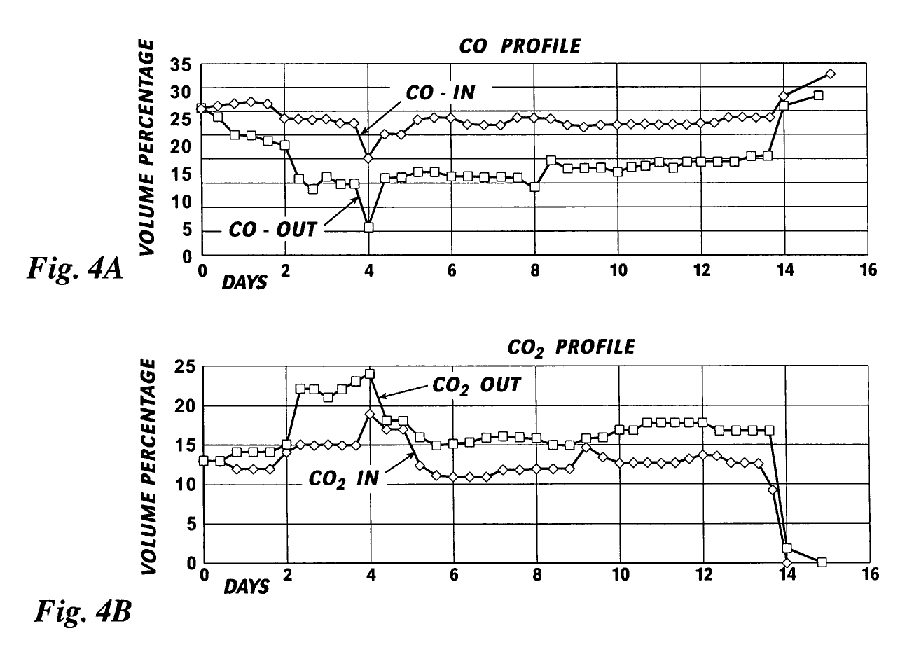

The necessity for CO₂ was confirmed by repeating the experiment with CO₂ in the feed gas. With CO₂, normal cell growth was established and maintained until the CO₂ supply was cut off on day 13. As can be seen in FIG. 4C, following cut-off, the cell concentration began decreasing. The experiment was terminated on day 15.

It was also observed (FIG. 4B) that CO₂ was always generated, not consumed, by the cells, establishing that CO₂ acted as a promoter of cell growth, but was not essentially consumed by the cells. In contrast, the CO profile (FIG. 4A) showed that CO was consumed. These results show that CO₂ is required in the feed gas although the cells can also produce CO₂ during fermentation. This anomaly has been observed in many clostridium fermentations, although a clear reason has note been established.

### Fermentation Products

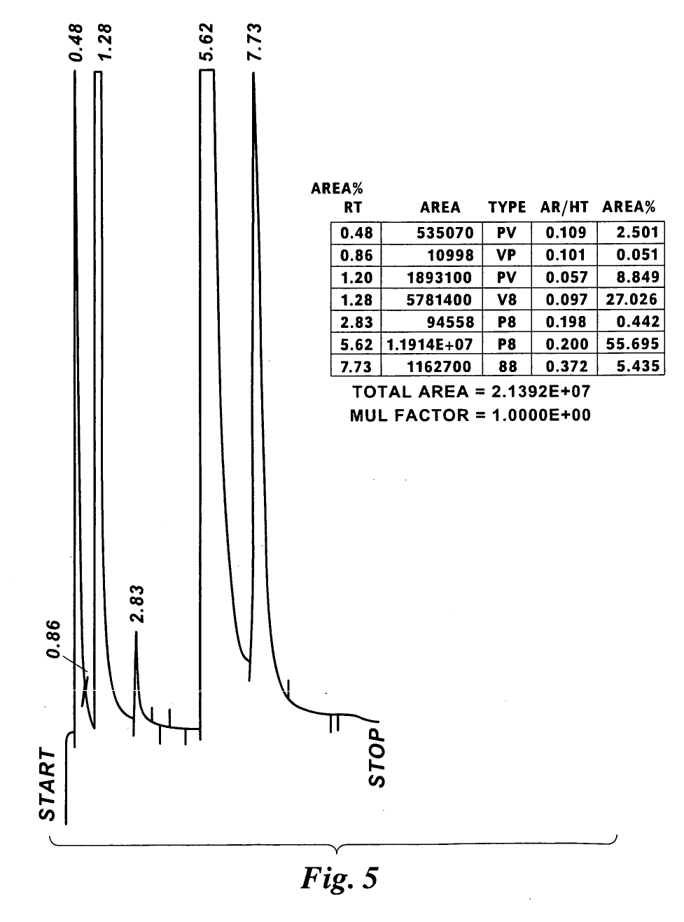

Material balance calculations were performed and showed that 90% of carbon was accounted for in the bioreactor. The maximum ethanol concentration observed in these initial experiments was 2.3 wt % at the end of the batch growth. In addition, acetate and low quantities of butanol were produced. An exemplary gas chromatogram showing the production of ethanol and butanol by P7 is presented in FIG. 5, where the peak at 1.28 represents ethanol, and the peak at 7.73 represents butanol.

### EXAMPLE 2

### Syngas Fermentations

The major known reactions in the biological conversion of syngas to ethanol and acetate by microbes are:

(i) Ethanol Synthesis

6CO+3H₂O→C₂H₅OH+4CO₂ (1)

6H₂+2CO₂→C₂H₅OH+3H₂O (2)

(iI) Acetate Synthesis

4CO+2H₂O→CH₃COOH+CO₂ (3)

4H₂+2 CO₂→CH₃COOH+2H₂O (4)

All experiments described herein were performed in a four-liter bubble column bioreactor made of plexiglass. The feed gas flow rate was 200 scan and consisted of CO (25%), CO₂ (15%) and N₂ (60%) blended from bottles. Hydrogen was not used in the initial study. Nutrients added to the bioreactor consisted of Pfennig's minerals and trace metals, vitamins, yeast extract, MES buffer and cysteine-sulfide as a reducing agent. Resazurin was added as an oxygen indicator. The pH of the media was initially 5.75 and, as the reaction proceeded, was controlled at 5.2. The reactor temperature was maintained at 37° C. using a hot water jacket. The inoculum was transferred to the bioreactor under sterile conditions. The cells were grown for at least 3 days in batch-mode, following which the bioreactor was switched to a continuous mode at 2 ml/min of product and feed flow rates.

### Analytical Procedure

Cell concentrations (in mg/ml) were determined at 660 nm using a spectrophotometer. Gas compositions were obtained by gas chromatography with a Hayesep-DB column connected to a Thermal Conductivity Detector using helium as the carrier gas. Liquid samples were centrifuged and headspace gases were analyzed for ethanol, butanol and acetic acid by the gas chromatograph using a solid phase microextraction technique. A Carbowax column connected to a flame ionization detector was used for the liquids.

### Results and Discussion

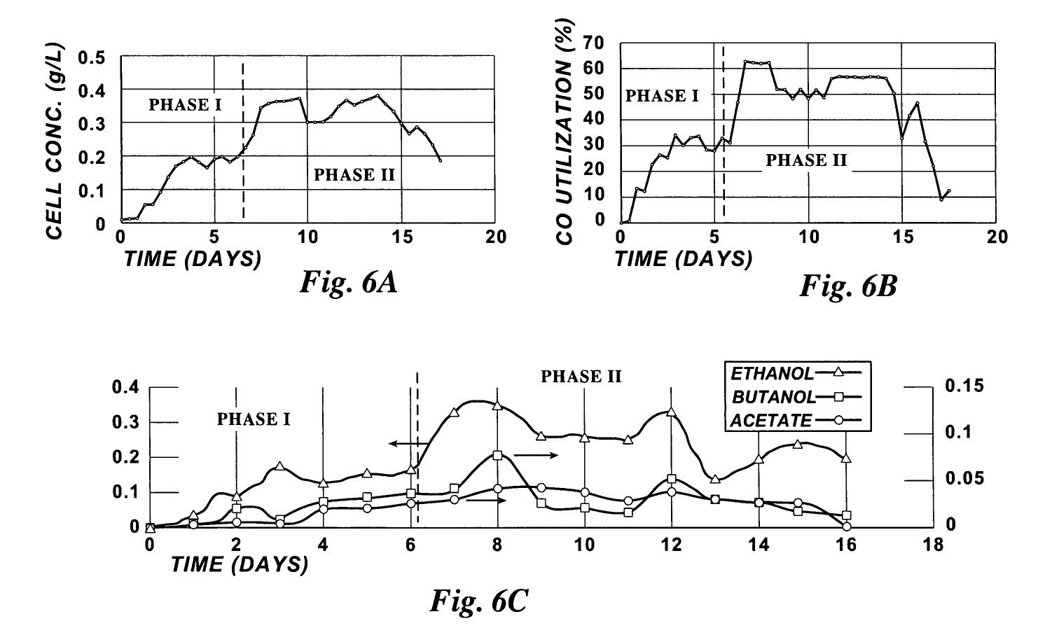

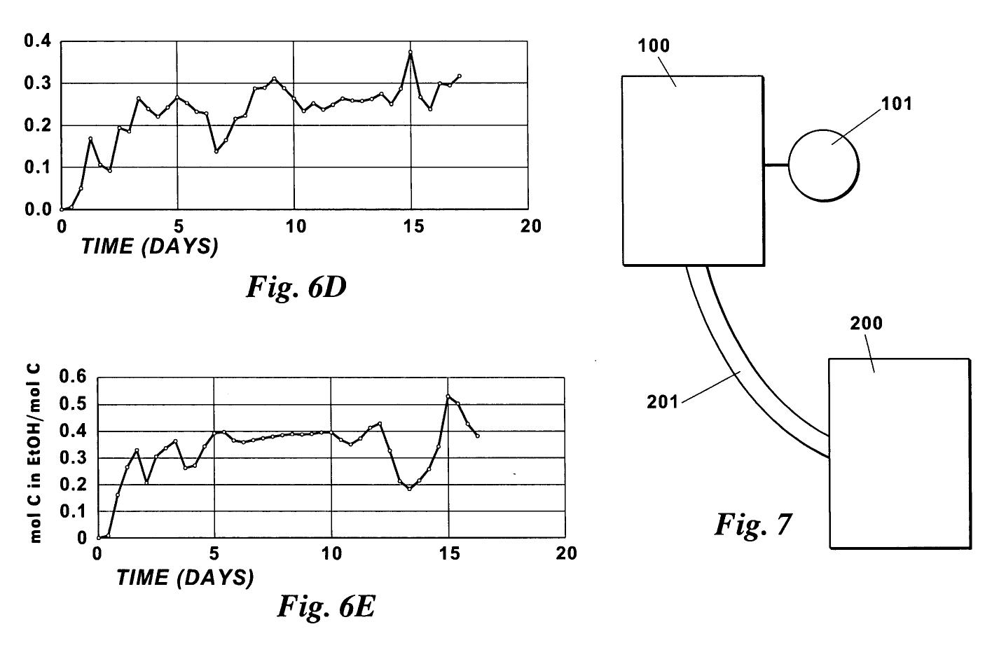

The experiments described herein lasted at least two weeks. FIGS. 6A and 6B show the cell concentration and CO utilization, respectively, with time. As can be seen, the cells started growing after a lag phase of about 1 day and stabilized at 0.2 g/L (shown in FIGS. 6A and 6B as Phase I). During this period, the CO utilization increased rapidly to 30% (FIG. 6B). The product profile is depicted in FIG. 6C. As can be seen, significant amounts of ethanol, acetate and butanol were produced, with ethanol being the primary product. At the end of 6 days, (i.e. at the onset of Phase II) the trace metal concentration in the bioreactor feed was doubled. As can be seen, 24 hours after doubling of the trace metal concentration, the cell concentration doubled to 0.35 g/L (FIG. 6A) and CO utilization reached 60% (FIG. 6B). During phase II, the ethanol concentration increased to 0.35 wt. %, and butanol and acetate concentrations increased to 0.075 wt. % and 0.035 wt. %, respectively (FIG. 6C). FIGS. 6D and 6E show the yields of cells and moles of carbon in ethanol per mole of CO, respectively, which were both independent of changes in the trace metal composition.

On day 13, the trace metal composition was again doubled, resulting in the initiation of cell death. The experiment was terminated on day 17.

The specific cell growth rate (μ) and yields (Y) at steady state are presented in Table 4.

TABLE 4
Cell growth rate (μ) and yields (Y) at steady state

| | |
|---|---|
| μ | 0.0025 min⁻¹ initial, 0.00044 min⁻¹ in continuous mode |
| Y_{ETOH/CO} | 0.33 mol/mol, based on carbon content |
| Y_{Butanol/CO} | 0.03 mol/mol, based on carbon content |
| Y_{Acetate/CO} | 0.04 mol/mol, based on carbon content |

The yield of ethanol from CO as compared to acetate and butanol is higher by 8 and 11 times respectively, establishing a high level of product selectivity and specificity of the new acetogen. However, up to 65% of CO was lost via the generation of CO₂ during the fermentation process. This loss can likely be minimized by the introduction of hydrogen gas supplements, which would result in increased utilization of CO₂ (and hence, CO), further increasing the yield of ethanol.

### Conclusions

This example demonstrates the anaerobic conversion of syngas to ethanol, acetate and butanol in continuous cultures of a newly isolated bacterium, ATCC BAA-624 (P7). This research is significant in terms of establishing the feasibility of the biochemical synthesis of ethanol fuels and other products from agricultural crops.

### References for Example 2

Klasson, K. T., I. L. Gaddy. (1992), Bioconversion of Synthesis Gas into Liquid Fuels. Enz. Micro. Tech., 14, 602-608.
Vega, J. L., E. C. Clausen, J. L. Gaddy. (1990). Design of Bioreactors for Coal Synthesis Gas Fermentations. Resources, Conservation and Recycling, 3, 149-160.
Phillips, J. R., E. C. Clausen, J. L. Gaddy (1994). Synthesis Gas as a Substrate for Biological Production of Fuels and Chemicals, App. Biochem. Biotech., 45/46, 145-156,
Barfield, B J., K. A. Kranzler, (1997). Economics of Biomass Conversion to Ethanol using Gasification with a Microbial Reactor. Report: Biosystems and Agricultural Eng., Oklahoma State University, Stillwater, Okla.
Taliaferro, C. M., F. P. Hoveland, B. B. Tucker, R. Totusek, R. D. Morrison, (1975).
Performance of Three Warm-Season Perennial Grasses and a Native Range Mixture as Influenced by N and P Fertilization. Agronomy, 67, 289-292,
Huhnke, R. L., W. Bowers. (1994). AGMACHS-Agricultural Field Machinery Cost Estimation Software. OSU Cooperative Extension Service, Oklahoma State University, Stillwater, Okla.

### EXAMPLE 3

### Further Optimization of Ethanol Production by P7

Optimization experiments showed the following:

1. The production of ethanol by P7 was enhanced two fold by increasing the level of iron in the standard medium. When the final concentration of iron was increased to 200 μM compared to the standard concentration of 20 μM, ethanol production increased from 20 mM to 40 mM under CO-limited conditions. When no iron was added to the standard medium, ethanol production was inhibited, similar to the effect of elimination of iron on the production of solvents in Clostridium acetobutylicum (McNeil and Kristiansen, 1985. The effect of medium composition on the acetone-butanol fermentation in continuous culture. Biotechnol. Bioeng. 29:383-387).

2. Controlling the culture pH at 5 (compared to the pH optimum for growth, 6), ethanol production was increased five fold. pH was adjusted using sterile anaerobic 1 N NaOH or HCl after monitoring pH using narrow range pH indicator strips (catolog no. 9582 EMD Chemicals, Inc., Gibbstown, N.J.). MES (20 g/L) was used as the primary buffer. At pH 6, 78 mM acetate and 15 mM butyrate were produced, but only 6 mM ethanol and 2 mM butanol. At pH 5, ethanol production increased to 32 mM and butanol to 5 mM, while the production of acids fell to 16 mM for acetate and 5 mM for butyrate, under CO-limited conditions. pH is known to significantly affect solvent production by clostridia (Jones and Woods, 1986. Acetone-butonal fermentation revisited. Microbiol. Rev. 50:484-524).

3. By optimizing these conditions (iron content and pH) and through culture adaptation P7 has been shown to produce 10.1 g/L of ethanol in batch culture, i.e. ethanol production in batch culture has been increased from 15 mM to 220 mM.

### EXAMPLE 4

### Direct Fermentation of Biomass.

P7 was used to ferment a slurry of 1% switchgrass. The results showed that P7 produced 1.3 mM ethanol and 7.4 mM acetic acid. This is comparable to results obtained in a control fermentation by Clostridium thermocellum, which produced 2.4 mM ethanol and 12 mM acetic acid. (See U.S. Pat. No. 4,292,406 to Ljungdahl et al, the entire contents of which are hereby incorporated by reference.) P7 is thus the first anaerobe described that can perform both an indirect and direct fermentation of lignocellulosic biomass.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A biologically pure culture of the microorganism Clostridium carboxidivorans having all of the identifying characteristics of ATCC No. BAA-624.

2. A composition for producing ethanol, comprising
a source of CO, and
Clostridium carboxidivorans.

3. The composition of claim 2, wherein said source of CO is syngas.

4. A method of producing ethanol, comprising the step of
combining a source of CO and Clostridium carboxidivorans under conditions which allow said Clostridium carboxidivorans to convert CO to ethanol.

5. A system for producing ethanol, comprising
a vessel in which a source of CO is combined with Clostridium carboxidivorans; and
a controller which controls conditions in said vessel which permit said Clostridium carboxidivorans to convert said CO to ethanol.

6. The system of claim 5, further comprising
a second vessel for producing syngas; and
a transport for transporting said syngas to said vessel, wherein said syngas serves as said source of CO.

7. A method for the direct fermentation of lignocellulosic biomass, comprising the step of
combining a source of lignocellulosic biomass and Clostridium carboxidivorans under conditions which allow said Clostridium carboxidivorans to directly ferment said lignocellulosic biomass to produce at least one of ethanol or acetic acid.
